(19) United States
(12) Patent Application Publication
(10) Pub. No.: US 2011/0039905 A1
(43) Pub. Date: Feb. 17, 2011

(54) BENZIMIDAZOLE DERIVATIVES AS CALCIUM CHANNEL BLOCKERS

(76) Inventors: Francis Hubler, Hegenheim (FR); Kurt Hilpert, Hofstetten (CH); Dorte Renneberg, Basel (CH)

Correspondence Address: HOXIE & ASSOCIATES LLC, 75 MAIN STREET , SUITE 301, MILLBURN, NJ, 07041, US

(21) Appl. No.: 12/989,443

(22) PCT Filed: Apr. 23, 2009
(86) PCT No.: PCT/IB2009/051668

§ 371 Date: Oct. 25, 2010

(30) Foreign Application Priority Data
Apr. 25, 2008 (IB) PCT/IB2008/051602

(51) Int. Cl. A61K 31/4184; C07D 235/14; A61P 9/00

(52) U.S. Cl. 514/394; 548/309.7

(57) ABSTRACT

The invention relates to compounds of formula (I)

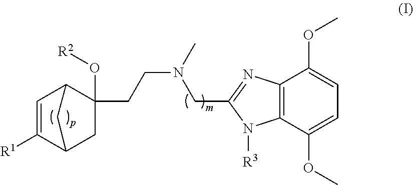

wherein
R¹ represents aryl, which is unsubstituted, or mono-, di-, or tri-substituted wherein the substituents are independently selected from the group consisting of (C₁₋₄)alkyl, (C₁₋₄)alkoxy, halogen, and trifluoromethyl;
R² represents hydrogen, or —CO—R²¹;
R²¹ represents (C₁₋₅)alkyl, (C₁₋₃)fluoroalkyl, or (C₃₋₆)cycloalkyl;
m represents the integer 2, or 3;
p represents the integer 2 or 3; and
R³ represents hydrogen, or (C₁₋₅)alkyl;
   and pharmaceutically acceptable salts of such compounds.

These compounds are useful as calcium channel blockers.

The present invention relates to novel benzimidazole derivatives and their use as potent calcium channel blockers in the treatment or prevention of chronic stable angina, hypertension, ischemia (renal and cardiac), cardiac arrhythmias including atrial fibrillation, cardiac hypertrophy, or congestive heart failure, to pharmaceutical compositions containing these derivatives and to processes for their preparation. The benzimidazole derivatives of the present invention may also be used, alone or in pharmaceutical compositions, for the treatment of renal diseases, diabetes and its complications, hyperaldosteronism, epilepsy, neuropathic pain, or cancer in humans and other mammals.

Many cardiovascular disorders have been associated with a ‘calcium overload’ resulting from an abnormal elevated calcium influx through the plasma membrane of cardiac and vascular smooth muscle cells. There are 3 major pathways through which extracellular calcium can enter these cells: 1) receptor-activated calcium channels, 2) ligand-gated calcium channels and 3) voltage-operated calcium channels (VOCs).

VOCs have been classified into 6 main categories: L (Long-lasting), T (Transient), N (Neuronal), P (Purkinje cells), Q (after P) and R (Remaining or Resistant).

L-type calcium channels are responsible for the inward movement of calcium that initiates contraction in cardiac and smooth muscle cells suggesting a putative application for blockers of these channels in the cardiovascular field. In this view, L-type calcium channel blockers have been used in clinic since the early 60s and are now recommended as a first line of treatment for systolic-diastolic hypertension and angina pectoris.

T-type calcium channels are found in various tissues such as coronary and peripheral vasculature, sinoatrial node and Purkinje fibres, brain, adrenal glands and in the kidney. This broad distribution suggests a T-type channel blocker to have a putative cardiovascular protection, to have en effect on sleep disorders, mood disorders, depression, migraine, hyperaldosteroneemia, preterm labor, urinary incontinence, brain aging or neurodegenerative disorders such as Alzheimers disease.

Mibefradil (Posicor®), the first L-type and T-type calcium channels blocker demonstrated a superior effect over calcium channel blockers, which target the L channel predominantly. Mibefradil was used for the treatment of hypertension and angina without showing negative side-effects often seen by L channel blockers like inotropy, reflex tachycardia, vasoconstrictive hormone release or peripheral edema. Additionally, mibefradil showed a potentially cardioprotective effect (Villame, Cardiovascular Drugs and Therapy 15, 41-28, 2001; Ramires, J Mol Cell Cardiol 30, 475-83, 1998), a renal protective effect (Honda, Hypertension 19, 2031-37, 2001), and showed a positive effect in the treatment of heart failure (Clozel, Proceedings Association American Physicians 111, 429-37, 1999). Despite the enormous demand for a compound of this profile, mibefradil was withdrawn from the market in 1998 (one year after its launch), due to unacceptable CYP 3A4 drug interactions. Moreover, ECG abnormalities (i.e. QT prolongations) and interaction with the MDR-1 mediated digoxin efflux were also reported (du Souich, Clin Pharmacol Ther 67, 249-57, 2000; Wandel, Drug Metab Dispos 28, 895-8, 2000).

There clearly is a demand for novel compounds, which act as T/L-type calcium channel blockers but have an improved safety profile with respect to mibefradil.

The compounds of the present invention are potent T/L channel blockers and therefore useful in diseases where both, T and L channels are involved.

i) A first aspect of the invention consists of benzimidazole derivatives of formula (I)

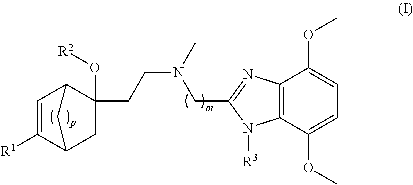

wherein
R¹ represents aryl, which is unsubstituted, or mono-, di-, or tri-substituted wherein the substituents are independently selected from the group consisting of (C₁₋₄)alkyl, (C₁₋₄)alkoxy, halogen, and trifluoromethyl;
R² represents hydrogen, or —CO—R²¹;
R²¹K represents (C₁₋₆)alkyl, (C₁₋₃)fluoroalkyl, or (C₃₋₆)cycloalkyl;
m represents the integer 2, or 3;
p represents the integer 2 or 3; and
R³ represents hydrogen, or (C₁₋₆)alkyl.

The following paragraphs provide definitions of the various chemical moieties for the compounds according to the invention and are intended to apply uniformly throughout the specification and claims, unless an otherwise expressly set out definition provides a broader or narrower definition.

The term “(C₁₋₆)alkyl” means a straight-chain or branched-chain alkyl group with 1 to 5 carbon atoms. Preferred are groups with 1 to 4 carbon atoms. The term “(C_{x-y})alkyl” (x and y being an integer) refers to a straight or branched chain alkyl group containing x to y carbon atoms. Examples of (C₁₋₆)alkyl groups are methyl, ethyl, n-propyl, isopropyl, n-butyl, sec.-butyl, tert.-butyl, isobutyl, n-pentyl, and isopentyl. Preferred are methyl, ethyl, n-propyl, and isopropyl. Most preferred is methyl. For the substituent R²¹, isopropyl is most preferred.

The term “(C₁₋₃)fluoroalkyl” means a straight-chain or branched-chain (C₁₋₃)alkyl group which is substituted with 1 to 7 fluorine atoms. Examples of (C₁₋₃)fluoroalkyl groups are trifluoromethyl, 2-fluoroethyl, 2,2-difluoroethyl, 2,2,2-trifluoroethyl, and pentafluoroethyl. Preferred are trifluoromethyl, 2,2,2-trifluoroethyl, and pentafluoroethyl. Most preferred is trifluoromethyl. For the substituent R²¹, 2,2,2-trifluoroethyl is most preferred.

The term “(C₃₋₆)cycloalkyl” means a saturated cyclic alkyl group with 3 to 6 carbon atoms. Examples of (C₃₋₆)cycloalkyl groups are cyclopropyl, cyclobutyl, cyclopentyl and cyclohexyl. For the substituent R²¹, cyclopropyl is most preferred.

The term “(C₁₋₆)alkoxy” means a group of the formula (C₁₋₆)alkyl-O— in which the term (C₁₋₆)alkyl has the previously given significance. The term “(C_{x-y})alkoxy” (x and y being an integer) refers to a straight or branched chain alkoxy group containing x to y carbon atoms. Examples of (C₁₋₆)alkoxy groups are methoxy, ethoxy, n-propoxy, isopropoxy, n-butoxy, isobutoxy, sec-butoxy and tert.-butoxy. Preferred are methoxy and ethoxy. The term “halogen” means fluoro, chloro, bromo or iodo, especially fluoro or chloro.

The term “aryl” means a phenyl or a naphthyl group. Preferred is a phenyl group. The aryl group may be unsubstituted, or mono-, di-, or tri-substituted wherein the substituents are independently selected from the group consisting of (C₁₋₄)alkyl, (C₁₋₄)alkoxy, halogen, and trifluoromethyl. In a sub-embodiment the aryl group is preferably unsubstituted. Examples of “aryl” groups are phenyl, naphthyl, 2-methylphenyl, 3-methylphenyl, 4-methylphenyl, 3,4-dimethylphenyl, 2,3-dimethylphenyl, 2,4-dimethylphenyl, 2,6-dimethylphenyl, 3,4-dimethylphenyl, 3,5-dimethylphenyl, 2-methoxyphenyl, 3-methoxyphenyl, 4-methoxyphenyl, 2,3-dimethoxyphenyl, 3,4-dimethoxyphenyl, 2-fluorophenyl, 3-fluorophenyl, 4-fluorophenyl, 3,4-difluorophenyl, 3-chlorophenyl, 2,3-dichlorophenyl, 3,4-dichlorophenyl, 2-trifluoromethylphenyl, 3-trifluoromethylphenyl, and 4-trifluoromethylphenyl. Preferred is phenyl

In the following, further embodiments of the invention are described:

ii) A further embodiment of the invention relates to compounds of formula (I) according to embodiment i), wherein the configuration of the bridged cyclohexene moiety is such that the R²—O— substituent and the bridge —(CH₂)ₚ— of the cyclohexene moiety are in cis relation (i.e. the absolute configuration is as depicted in either formula (I_{E1}) or formula (I_{E2}) below).
iii) A further embodiment of the invention relates to compounds of formula (I) according to embodiment i), wherein the absolute configuration is as depicted in formula (I_{E1})

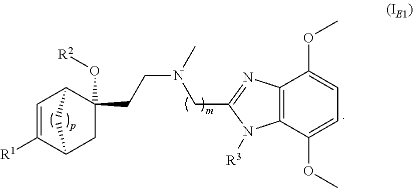

iv) A further embodiment of the invention relates to compounds of formula (I) according to embodiment i), wherein the absolute configuration depicted is as in formula (I_{E2})

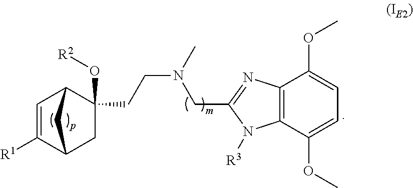

v) A further embodiment of the invention relates to compounds of formula (I) according to any one of embodiments i) to iv), wherein R¹ represents unsubstituted phenyl.
vi) A further embodiment of the invention relates to compounds of formula (I) according to embodiments i) to v), wherein p represents the integer 2.
vii) A further embodiment of the invention relates to compounds of formula (I) according to embodiments i) to v), wherein p represents the integer 3.
viii) A further embodiment of the invention relates to compounds of formula (I) according to any one of embodiments i) to vii), wherein R² represents —CO—R²¹.
ix) A further embodiment of the invention relates to compounds of formula (I) according to any one of embodiments i) to viii), wherein R²¹ represents (C₁₋₆)alkyl, or (C₃₋₆)cycloalkyl.
x) A further embodiment of the invention relates to compounds of formula (I) according to any one of embodiments i) to ix), wherein R²¹ represents (C₁₋₆)alkyl (especially isopropyl).
xi) A further embodiment of the invention relates to compounds of formula (I) according to any one of embodiments i) to vii), wherein R² represents hydrogen.
xii) A further embodiment of the invention relates to compounds of formula (I) according to any one of embodiments i) to xi), wherein m represents the integer 3.
xiii) A further embodiment of the invention relates to compounds of formula (I) according to any one of embodiments i) to xii), wherein R³ represents hydrogen.
xiv) A further embodiment of the invention relates to compounds of formula (I) according to any one of embodiments i) to xii), wherein R³ represents (C₁₋₆)alkyl (especially methyl).

The compounds of formula (I) contain stereogenic or asymmetric centers, such as asymmetric carbon atoms. The compounds of formula (I) may thus be present as mixtures of stereoisomers or preferably as pure stereoisomers. Mixtures of stereoisomers may be separated in a manner known to a person skilled in the art.

Preferred compounds of formula (I) are selected from the group consisting of:
(1R,2R,4R)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol;
(1S,2S,4S)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol; and
(1R*,5R*,6R*)-6-(2-{[3-(4,7-Dimethoxy-1H-benzoimidazol-2-yl)-propyl]-methyl-amino}-ethyl)-8-phenyl-bicyclo[3.2.2]non-8-en-6-ol.

Additionally, further preferred compounds of formula (I) according to embodiment i) are selected from the group consisting of:
Isobutyric acid (1R,2R,4R)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester;
Isobutyric acid (1S,2S,4S)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester; and
Isobutyric acid (1R*,5R*,6R*)-6-(2-{[3-(4,7-dimethoxy-1H-benzoimidazol-2-yl)-propyl]-methyl-amino}-ethyl)-8-phenyl-bicyclo[3.2.2]non-8-en-6-yl ester.

The relative configuration of stereoisomers is denoted as follows: for example, isobutyric acid (1R*,5R*,6R*)-6-(2-{[3-(4,7-dimethoxy-1H-benzoimidazol-2-yl)-propyl]-methyl-amino}-ethyl)-8-phenyl-bicyclo[3.2.2]non-8-en-6-yl ester denominates
isobutyric acid (1R,5R,6R)-6-(2-{[3-(4,7-dimethoxy-1H-benzoimidazol-2-yl)-propyl]-methyl-amino}-ethyl)-8-phenyl-bicyclo[3.2.2]non-8-en-6-yl ester,
isobutyric acid (1S,5S,6S)-6-(2-{[3-(4,7-dimethoxy-1H-benzoimidazol-2-yl)-propyl]-methyl-amino}-ethyl)-8-phenyl-bicyclo[3.2.2]non-8-en-6-yl ester, or mixtures of these two enantiomers.

Where the plural form is used for compounds, salts, pharmaceutical compositions, diseases and the like, this is intended to mean also a single compound, salt, or the like.

Any reference to a compound of formulae (I), (I_{E1}), and/or (I_{E2}) is to be understood as referring also to the salts (and especially the pharmaceutically acceptable salts) of such compounds, as appropriate and expedient.

The term “pharmaceutically acceptable salts” refers to non-toxic, inorganic or organic acid and/or base addition salts. Reference can be made to “Salt selection for basic drugs”, Int. J. Pharm. (1986), 33, 201-217.

The compounds of formulae (I), (I_{E1}), and/or (I_{E2}) and their pharmaceutically acceptable salts can be used as medicaments, e.g. in the form of pharmaceutical compositions for enteral or parenteral administration.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula (I), or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The compounds of formula (I), or a pharmaceutically acceptable salt thereof, are useful in the preparation of a medicament
for the treatment or prevention of chronic stable angina, hypertension, ischemia (renal and cardiac), cardiac arrhythmias including atrial fibrillation, cardiac hypertrophy, or congestive heart failure.

The compounds of formula (I), or a pharmaceutically acceptable salt thereof, are further also useful in the preparation of a medicament for the following disease groups alone or in any combination:
for the treatment of renal diseases, diabetes and its complications, hyperaldosteronism, epilepsy, neuropathic pain, or cancer in humans and other mammals;
for use as anti-fibrillatory agent, anti-asthmatic agent, anti-atherosclerotic agent, additive to cardioplegic solutions for pulmonary bypasses, adjunct to thrombolytic therapy, as antiaggregant agent, or as agent for the treatment of unstable angina;
for the treatment or prophylaxis of hypertension, especially portal hypertension, hypertension secondary to treatment with erythropoietin and low renin hypertension;
for use in hypoxic or ischemic diseases, or as anti ischemic agent for the treatment of e.g. cardiac, renal and cerebral ischemia and reperfusion (e.g. occurring after cardiopulmonary bypass surgery), coronary and cerebral vasospasm and the like, therapy for peripheral vascular diseases (e.g. Raynaud's disease, intermittent claudication, Takayashus disease), sickle cell disease including initiation and/or evolution of the pain crisis;
for the treatment or prophylaxis of disorders related to renal, glomerular and mesangial cell function, including acute and chronic renal failure, diabetic nephropathy, hypertension-induced nephropathy, glomerular injury, renal damage related to age or dialysis, nephrosclerosis, nephrotoxicity related to imaging and contrast agent and to cyclosporine, renal ischemia, primary vesicoureteral reflux, or glomerulosclerosis;
for use in therapy for myocardial infarction, treatment of cardiac hypertrophy, primary and secondary pulmonary hypertension, therapy for congestive heart failure including inhibition of fibrosis, inhibition of left ventricular dilatation, remodelling and dysfunction, or restenosis following angioplasty or stenting;
for the treatment of endotoxemia or endotoxin shock, or hemorrrhagic shock;
for the treatment of sexual dysfunction in both men (erectile dysfunction e.g. due to diabetes mellitus, spinal cord injury, radical prostatectomy, psychogenic etiology and other causes) and women by improving blood flow to the genitalia, especially corpus cavernosum;
for the prevention and/or reduction of cancer or end-organ damage associated with cell proliferation;
for therapy of metabolic disorders or chronic inflammatory diseases, insulin-dependent and non insulin-dependent diabetes mellitus and their complications (e.g. neuropathy, retinopathy), hyperaldosteronism, bone remodelling, psoriasis, arthritis, rheumatoid arthritis, osteoarthritis sarcoidosis, or eczematous dermatitis;
for the treatment of hepatotoxicity and sudden death, early and advanced liver disease and injury including attendant complication (e.g. hepatotoxicity, fibrosis, cirrhosis), deleterious consequences of tumors such as hypertension resulting from hemangiopericytoma, spastic diseases of the urinary tract and/or bladder, hepatorenal syndrome, immunological diseases involving vasculitis such as lupus, systemic sclerosis, mixed cryoglobulinemia, fibrosis associated with renal dysfunction and hepatotoxicity;
for use in gastrointestinal diseases such as ulcerative colitis, Crohn's disease, gastric mucosal damage, ulcer inflammatory bowel disease and ischemic bowel disease, gall bladder or bile duct-based diseases such as cholangitis, pancreatitis, regulation of cell growth, beginning prostatic hypertrophy, or transplantation, or for use as anti-diarrheal agent;
for the treatment of disorders involving bronchoconstriction or disorders of chronic or acute inflammation such as obstructive pulmonary disease and adult distress syndrome;
for the alleviation of pain including neuropathic pain, peripheral pain and pain associated with cancer such as pain associated with prostate cancer or bone-cancer;
for the treatment of central nervous system vascular disorders such as stroke, transient ischemic attacks, migraine and subarachnoid hemorrhage, central nervous system behavioural disorders, treatment of dementia including Alzheimer's dementia, senile dementia and vascular dementia, epilepsy, or sleep disorders; or
for reduction of general morbidity and/or mortality as a result of above utilities.

The present invention also relates to a method for the prevention or treatment of a disease or disorder mentioned herein comprising administering to a subject a pharmaceutically active amount of a compound of formula (I).

Furthermore, the compounds of the formula (I) may also be used favourably in combination with one or more agents selected from lipid lowering agents such as statins, anticoagulants such as coumarins, antithrombotic agents such as clopidogrel, β-blockers, and other cardioprotective agents.

Besides, any preferences indicated for the compounds of formula (I) (whether for the compounds themselves, salts thereof, compositions containing the compounds or salts thereof, uses of the compounds or salts thereof, etc.) apply mutatis mutandis to compounds of formulae (I_{E1}), and/or (I_{E2}) and vice versa.

### Preparation of Compounds of Formula (I):

A further aspect of the invention is a process for the preparation of compounds of formulae (I) of the present invention. The compounds obtained may also be converted into pharmaceutically acceptable salts thereof in a manner known per se.

In general, all chemical transformations can be performed according to well-known standard methodologies as described in the literature or as described in the procedures as summarized in Schemes 1 to 3 below. If not indicated otherwise, the generic groups or integers R¹, R², R³, p, and m are as defined for formula (I). Other abbreviations used are defined in the experimental section. In some instances the generic groups R¹, R², R³ might be incompatible with the assembly illustrated in the schemes below and so will require the use of protecting groups (PG). The use of protecting groups is well known in the art (see for example “Protective Groups in Organic Synthesis”, T. W. Greene, P. G. M. Wuts, Wiley-Interscience, 1999). For the purposes of this discussion, it will be assumed that such protecting groups as necessary are in place.

Compounds of formula (I) are prepared following the procedures outlined in Scheme 1 below.

Compounds of formula (I) wherein R² represents H can be prepared by saponification of the ester K using standard basic conditions such as LiOH or NaOH in solvents like ethanol, methanol, THF or water at rt, or standard acidic conditions such as aq. HCl or TFA in solvents like ethanol, methanol, THF, DCM, or water at rt to yield the acid derivatives 1.1. This acid is then coupled with benzimidazole derivatives BB to give the amide derivatives 1.2 using standard coupling reagents such as EDC, HOBt or PyBOP in the presence of a base such as NEt₃ or DIPEA and in solvents such as THF, DCM or DMF, preferably at rt. The amide 1.2 is then reduced to give the desired compounds of formula (I) wherein R² represents H using standard reducing agents like LiAlH₄ or Red-Al in adequate solvents such as toluene at temperatures between 0° C. to rt.

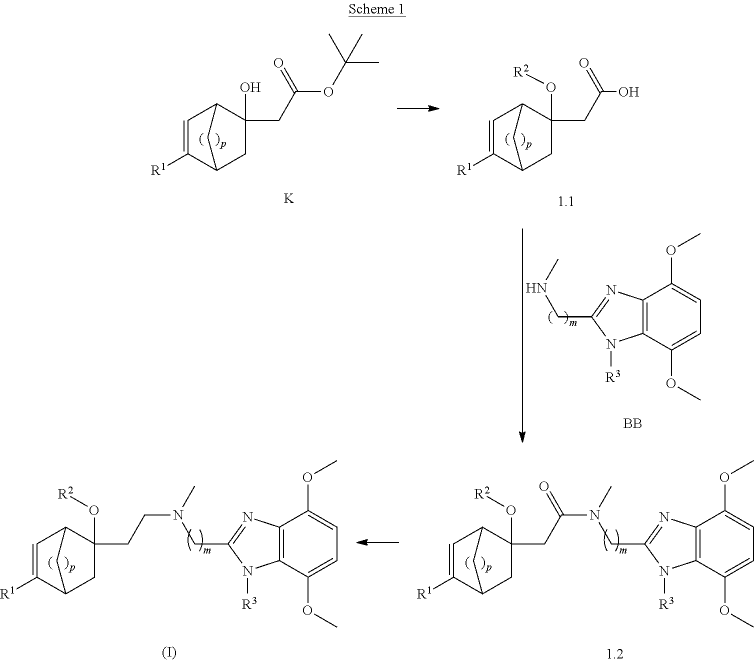

Alcohols of compounds of formula (I) wherein R² represents H can be acylated using standard reagents such as acid chlorides, acid anhydrides, chloroformates, isocyanates, or carbamoylchlorides, if necessary in presence of a Lewis acid such as MgBr₂, or in presence of a base such as NEt₃ in inert solvents such as DCM or THF at temperatures between 0° C. and 65° C. to give compounds of formula (I) wherein R² represents —COR²¹.

The key intermediates K are prepared according to Scheme 2. Diketones 2.1 and mono protected ketones 2.2 can be prepared according to known procedures (Can. J. Chem. 1992, 70, 974-980, Can. J. Chem. 1968, 46, 3713-17, JOC 1978, 43, 4648-4650).

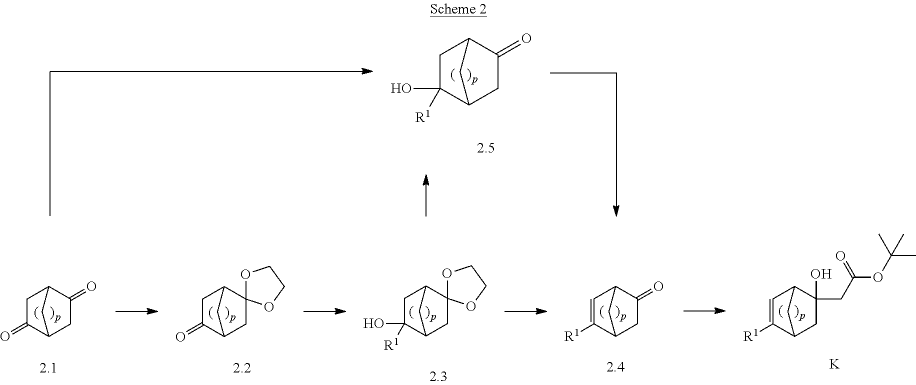

Alkylation of the ketone 2.2 with nucleophiles like Grignard reagents or lithiated reagents (prepared from the corresponding bromo compound with e.g. butyllithium using standard reaction conditions) such as phenylmagnesiumbromide, in adequate solvents like Et₂O or THF at temperatures between −78° C. and rt yields the alcohols 2.3.

Hydrolysis of the ketal of alcohol derivative 2.3 and subsequent elimination of water using standard dehydration reagents and procedures such as TsOH in adequate solvents such as acetone preferably at rt leads to the ketone 2.4.

Alternatively, this deprotection/elimination reaction can be performed in two steps. The ketal of alcohol derivative 2.3 is hydrolyzed as described above using protic conditions such as TsOH in solvents such as acetone at rt to yield the ketone derivative 2.5. The elimination of water can be performed using standard conditions such as Ms-Cl in presence of a base like NEt₃ and in adequate solvents like DCM at temperatures between 0° C. and rt or using the Burgess reagent in adequate solvents like THF at temperatures between 0° C. and rt to lead to ketone derivatives 2.4.

In another variation the diketone 2.1 can be selectively mono-alkylated directly to ketone derivative 2.5 by appropriate nucleophiles like Grignard reagents in standard solvents like Et₂O or THF at temperatures about 0° C. The elimination of water can then be performed applying the same conditions as mentioned above.

Ketone derivatives 2.4 are transformed to the desired key intermediates K by addition of nucleophiles such as Grignard reagents or lithiated alkyl groups such as lithiated tert.-butylacetate (prepared in situ using tert.-butyl bromoacetate, n-butyllithium and DIPA at temperatures of −50° C. in an adequate mixture of solvents such as toluene-THF or hexane-THF) at temperatures between −50° C. and rt.

The synthesis of the benzimidazole derivatives BB (Scheme 1) is outlined in Scheme 3. A suitably substituted dianiline derivative 3.1, which is synthesized e.g. from 1,4-dimethoxy-2,3-dinitro-benzene (Eur. J. Org. Chem. 2006, 2786-2794) according to standard procedures or following the methods given in the experimental part below, is coupled to an accordingly protected, commercially available N-alkylamino-alkanoic acid derivative using standard coupling reagents and conditions such as EDC/HOBt in presence of a base such as DIPEA, NEt₃, DMAP in solvents like THF, DCM at rt to give the aniline derivatives 3.2, wherein PG refers to an amino protecting group such as Cbz or BOC. Heating of 3.2, preferably under microwave conditions to about 150° C., neat or in appropriate solvents such as toluene or acetic acid leads to the protected aminoalkyl benzimidazole derivatives 3.3. Optionally, in case R³ is alkyl, the substituent can be introduced using standard reactions such as alkylation with an appropriate alkyl halogenide in presence of a base like NaH or K₂CO₃ in a solvent like acetone, DMF or THF at temperatures of about 0° C. Deprotection using standard deprotection procedures (hydrogenation for PG=Cbz; TFA or HCl for PG=BOC) gives the desired aminoalkyl benzimidazole derivatives BB.

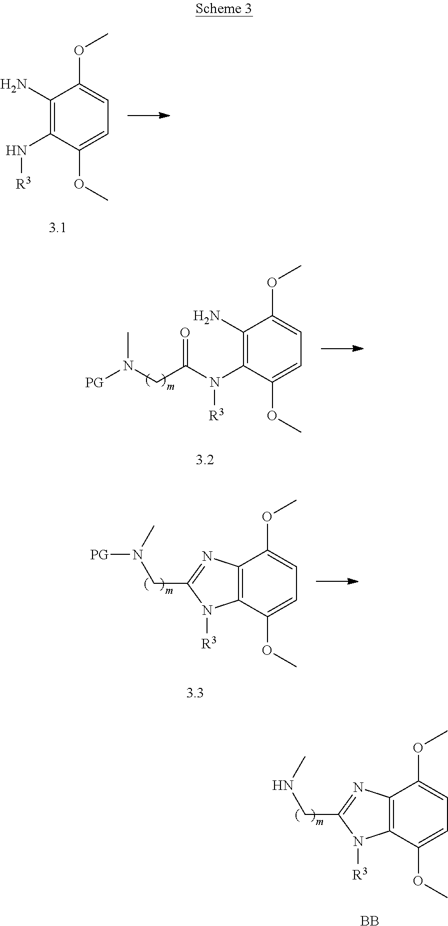

Whenever the compounds of formula (I) are obtained in the form of mixtures of enantiomers, the enantiomers can be separated using methods known to one skilled in the art: e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in presence or absence of an amine such as NEt₃, diethylamine) and eluent B (Hex), at a flow rate of 0.8 to 150 mL/min.

### Experimental Part

The following examples illustrate the invention but do not at all limit the scope thereof.

All temperatures are stated in ° C. Compounds are characterized by ¹H-NMR (400 MHz) or ¹³C-NMR (100 MHz) (Bruker; chemical shifts are given in ppm relative to the solvent used; multiplicities: s=singlet, d=doublet, t=triplet, q=quartett, p=pentuplet, hex=hexet, hept=heptet, m=multiplet, br=broad, coupling constants are given in Hz); by LC-MS (Finnigan Navigator with HP 1100 Binary Pump and DAD, column: 4.6×50 mm, Zorbax SB-AQ, 5 μm, 120 Å, gradient: 5-95% acetonitrile in water, 1 min, with 0.04% trifluoroacetic acid, flow: 4.5 mL/min), t_{R} is given in min; by TLC (TLC-plates from Merck, Silica gel 60 F₂₅₄); or by melting point. Compounds are purified by preparative HPLC (column: X-terra RP18, 50×19 mm, 5 μm, gradient: 10-95% acetonitrile in water containing 0.5% of formic acid) or by column chromatography on silica gel. Racemates can be separated into their enantiomers by preparative HPLC (preferred conditions: Daicel, ChiralCel OD 20×250 mm, 10 μm, 4% ethanol in hexane, flow 10-20 mL/min).

### ABBREVIATIONS

### As Used Herein or in the Description Above

aq. aqueous
Ac acetyl
anh. anhydrous
BOC tert.-butoxycarbonyl
BSA bovine serum albumin
Bu butyl
Cbz benzyloxycarbonyl
CC column chromatography on silica gel
Burgess reagent (methoxycarbonylsulfamoyl)triethylammonium hydroxide
d day(s)
DCM dichloromethane
dil. diluted
DIPA diisopropylamine
DIPEA diisopropyl-ethylamine, Hünig's base, ethyl-diisopropylamine
DMAP dimethylaminopyridine
DMF dimethylformamide
DMSO dimethylsulfoxide
EDC N-(3-dimethylaminopropyl)-N′-ethylcarbodiimide
eq. equivalent(s)
Et ethyl
EtOAc ethyl acetate
EtOH ethanol
Et₂O diethyl ether
h hour(s)
Hept heptane
Hex hexane
HOBt 1-hydroxybenzotriazole
HPLC high performance liquid chromatography
LC-MS liquid chromatography—mass spectrometry
Me methyl
MeCN acetonitrile
MeOH methanol
min minute(s)
Ms methanesulfonyl
NEt₃ triethylamine
Pd/C palladium on carbon
prep. preparative
PyBOP benzotriazole-1-yl-oxy-tris-pyrrolidino-phosphonium hexafluorophosphate
sat. saturated
tert.- tertiary (tert.-butyl=t-butyl=tertiary butyl)
TFA trifluoroacetic acid
THF tetrahydrofuran
TLC thin layer chromatography
Red-Al sodium-bis(2-methoxyethoxy)aluminumhydride
rt room temperature
t_{R} retention time
Ts para-toluenesulfonyl
TsOH para-toluenesulfonic acid

### Preparation of Intermediates

### General Procedures for the Preparation of Key Intermediates K:

Key intermediates K1A and K2A which are bicyclo[2.2.2]oct-5-en-2-yl or bicyclo[3.2.2]non-8-en-6-yl derivatives are obtained as a mixture between the major racemate having the relative configuration (R*,R*,R*) (i.e. the bridge —(CH₂)ₚ— of the cyclohexene moiety is cis to the group —OR² being hydroxy) and the minor racemate having the relative configuration (R*,S*,R*) or (R*,R*,S*), respectively (i.e. the bridge —(CH₂)ₚ— (wherein p represents 2 or 3, repectively) of the cyclohexene moiety is trans to the group —OR² being hydroxy). The major and the minor racemates can be separated as described for key intermediate K1A in procedure A1.5. If not stated otherwise only the major racemate is isolated and used in the preparation of the examples below.

### K1A: rac-(1R*,2R*,4R*)-(2-Hydroxy-5-phenyl-bicyclo[2.2.2]oct-5-en-2-yl)-acetic acid tert.-butyl ester

### K1A.1 (Procedure A1.1): rac-(1R*,4R*)-Bicyclo[2.2.2]octane-2,5-dione

25 mL of 2-(trimethylsilyloxy)-1,3-cyclohexadiene and 13 mL of α-acetoxyacrylonitrile were mixed and heated at 150° C. in a closed vessel for 22 h. The obtained dark orange viscous oil was dissolved in 200 mL of MeOH. After dropwise addition of a solution of 2.2 g of sodium methoxide in 150 mL of MeOH the reaction mixture was stirred for 3 h at rt, poured into ice/water and extracted with DCM. The organic phases were concentrated in vacuo and the crude residue was purified by CC with EtOAc-Hept (1:2) to yield 7.9 g of rac-(1R*,4R*)-bicyclo[2.2.2]octane-2,5-dione.

LC-MS: t_{R}=0.44 min.

### K1A.2 (Procedure A1.2): rac-(1R*,4R*)-Spiro[bicyclo[2.2.2]octane-2,2″-[1,3]dioxolan]-5-one

To 4.0 g of rac-(1R*,4R*)-bicyclo[2.2.2]octane-2,5-dione (intermediate K1A.1), dissolved in 120 mL of toluene, 1.7 mL of ethylene glycol and 0.27 g of TsOH were added and the solution was heated under vigorous stirring to reflux for 3.5 h. The reaction mixture was cooled to rt, quenched with saturated aq. NaHCO₃, extracted with Et₂O, and the organic phase was evaporated. The crude product was purified by CC with Hex-EtOAc (7:3) to yield 2.41 g of rac-(1R*,4R*)-spiro[bicyclo[2.2.2]octane-2,2′-[1,3]dioxolan]-5-one as yellow oil.

LC-MS: t_{R}=0.64 min; [M+H+CH₃CN]⁺: 224.35.

### K1A.3 (Procedure A1.3): Mixture of rac-(7R*,8R*,10R*) and rac-(7R*,8S*,10R*)-7,10-(1,2-Ethylen)-8-phenyl-1,4-dioxa-spiro[4.5]decan-8-ol

To a solution of 2.41 g of rac-(1R*,4R*)-spiro[bicyclo[2.2.2]octane-2,2′-[1,3]dioxolan]-5-one (intermediate K1A.2) in 80 mL Et₂O, 14.5 mL phenylmagnesium bromide solution (1 M in Et₂O) was added dropwise over 10 min. The reaction mixture was stirred for 4 h at rt. Then, the mixture was quenched carefully with ice, 8 mL 2N HCl were added and the phases were separated. The organic phase was evaporated and the crude product was purified by CC with Hept-EtOAC (7:3) to give 0.37 g of 7,10-(1,2-ethylen)-8-phenyl-1,4-dioxa-spiro[4.5]decan-8-ol as colorless oil. (Separation of the diastereomers by CC is possible but was performed only if stated.)

LC-MS: t_{R}=0.84 min; [M−H₂O+H]⁺: 243.34.

### K1A.4 (Procedure A1.4): rac-(1R*,4R*)-5-Phenyl-bicyclo[2.2.2]oct-5-en-2-one

To a solution of 0.54 g of 7,10-(1,2-ethylen)-8-phenyl-1,4-dioxa-spiro[4.5]decan-8-ol (intermediate K1A.3) in 20 mL acetone was added 200 mg of TsOH and then the mixture was stirred for 2 d at rt. The reaction mixture was quenched with sat. aq. NaHCO₃, extracted with EtOAC and the organic phase was evaporated. The crude product was purified by CC with Hept-EtOAC (7:3) to give 0.34 g of rac-(1R*,4R*)-5-phenyl-bicyclo[2.2.2]oct-5-en-2-one as colorless oil.

LC-MS: t_{R}=0.93 min; [M+H+CH₃CN]⁺: 240.11.

### K1A.5 (Procedure A1.5): rac-(1R*,2R*,4R*)-(2-Hydroxy-5-phenyl-bicyclo[2.2.2]oct-5-en-2-yl)-acetic acid tert.-butyl ester and rac-(1R*,2S*,4R*)-(2-hydroxy-5-phenyl-bicyclo[2.2.2]oct-5-en-2-0)-acetic acid tert.-butyl ester

To a solution of 0.51 mL of DIPA in 0.5 mL THF 2.2 mL of n-butyllithium (1.6M in Hex) were added dropwise at −20° C. After 10 min, 0.5 mL of toluene were added and the solution was stirred for 30 min. The mixture was cooled to −50° C., 0.73 mL of tert.-butyl acetate were added and stirring was continued for 1 h at −50° C. Then 0.32 g of rac-(1R*,4R*)-5-phenyl-bicyclo[2.2.2]oct-5-en-2-one (intermediate K1A.4) dissolved in 1 mL of THF was added and the solution was stirred at −50 to −20° C. over 2.5 h. The reaction mixture was poured on ice/aq. HCl, the organic phase was separated, washed and evaporated. The crude reaction product was purified by CC with Hept-EtOAc (9:1) to yield 0.30 g of the major racemate, rac-(1R*,2R*,4R*)-2-hydroxy-5-phenyl-bicyclo[2.2.2]oct-5-en-2-yl)-acetic acid tert.-butyl ester, as white solid and 0.07 g of the minor racemate, rac-(1R*,2S*,4R*)-2-hydroxy-5-phenyl-bicyclo[2.2.2]oct-5-en-2-yl)-acetic acid tert.-butyl ester, as colorless oil.

LC-MS (major racemate): t_{R}=1.06 min; [M−(CH₃)₃—H₂O—+H]⁺: 241.11.

LC-MS (minor racemate): t_{R}=1.05 min; [M+H]⁺: 315.18.

### K1A.6: (1S,2S,4S)-(2-Hydroxy-5-phenyl-bicyclo[2.2.2]oct-5-en-2-yl)-acetic acid tert.-butyl ester and (1R,2R,4R)-(2-Hydroxy-5-phenyl-bicyclo[2.2.2]oct-5-en-2-yl)-acetic acid tert.-butyl ester

rac-(1R*,2R*,4R*)-(2-Hydroxy-5-phenyl-bicyclo[2.2.2]oct-5-en-2-yl)-acetic acid tert.-butyl ester was separated into the respective enantiomers using prep. chiral HPLC (column: Daicel ChiralPak AD-H, 20×250 mm, 5 μm; Hex/EtOH 95:5, flow 16 mL/min)

Chiral analytic HPLC (Daicel ChiralPak AD-H, 4.6×250 mm, 5 μm; Hex/EtOH 95:5, flow 0.8 mL/min):

Enantiomer A: t_{R}=6.70 min.

Enantiomer B: t_{R}=7.93 min.

### K2A: rac-(1R*,5R*,6R*)-(6-Hydroxy-8-phenyl-bicyclo[3.2.2]non-8-en-6-yl)-acetic acid tert.-butyl ester

### K2A.1 (Procedure A1.6): Mixture of rac-(1R*,5R*,8R*) and rac-(1R*,5R*,8S*)-8-hydroxy-8-phenyl-bicyclo[3.2.2]nonan-6-one

To a suspension of 1.4 g of rac-(1R*,5R*)-bicyclo[3.2.2]nonane-6,8-dione (synthesized according to known procedures: Can. J. Chem. 1968, 46, 3713-3717) in 45 mL of Et₂O 10.3 mL of phenylmagnesiumbromide solution (1 M in THF) were added successively during 15 min at 0° C. and the mixture was stirred for 2 h at rt. The reaction mixture was then cooled to 0° C., quenched with ice-water, acidified with aq. HCl and extracted with Et₂O. The organic phase was washed with brine, dried over MgSO₄ and concentrated in vacuo to obtain the crude title compound as yellow oil.

LC-MS: t_{R}=0.79 min; [M+H+CH₃CN]⁺: 272.33.

### K2A.2 (Procedure A1.7): rac-(1R*,5R*)-8-Phenyl-bicyclo[3.2.2]non-8-en-6-one

The above crude 8-hydroxy-8-phenyl-bicyclo[3.2.2]nonan-6-one (intermediate K2A.1) was dissolved in 55 mL of acetone, 1.7 g of TsOH were added and the mixture was stirred at rt overnight. Another 3.5 g of TsOH were added and stirring was continued for further 5 h. The reaction mixture was then diluted with EtOAc, the organic phase was washed with sat. aq. NaHCO₃ and evaporated. The crude material was purified by CC with Hept-EtOAc (4:1) to yield 0.9 g of rac-(1R*,5R*)-8-phenyl-bicyclo[3.2.2]non-8-en-6-one as yellowish oil.

LC-MS: t_{R}=0.99 min; [M+H]⁺: 213.11.

### K2A.3: rac-(1R*,5R*,6R*)-(6-Hydroxy-8-phenyl-bicyclo[3.2.2]non-8-en-6-yl)-acetic acid tert.-butyl ester

Prepared from rac-(1R*,5R*)-8-phenyl-bicyclo[3.2.2]non-8-en-6-one (intermediate K2A.2) using procedure A1.5.

LC-MS (major racemate): t_{R}=1.11 min; [M−(CH₃)₃—H₂O+H]⁺: 254.02.

### BB. [3-(4,7-Dimethoxy-1H-benzoimidazol-2-yl)-propyl]-methyl-amine

### BB.1 3,6-Dimethoxy-benzene-1,2-diamine

3,6-Dimethoxy-benzene-1,2-diamine was synthesized by dissolving 6.0 g of 1,4-dimethoxy-2,3-dinitro-benzene (Eur. J. Org. Chem. 2006, 2786-2794) in 220 mL EtOH, evacuating 3 times with N₂ and adding 600 mg of 10% Pd/C. The reaction was stirred under a H₂ atmosphere (balloon). Another 300 mg of 10% Pd/C were added after 2 days and the mixture was stirred for another 24 h. Filtration over a pad of celite and washing with EtOH and EtOAc yielded after concentration in vacuo 4.3 g of 3,6-dimethoxy-benzene-1,2-diamine as black solid.

LC-MS: t_{R}=0.48 min; [M+H]⁺: 169.09.

### BB.2 [3-(2-Amino-3,6-dimethoxy-phenylcarbamoyl)-propyl]-methyl-carbamic acid benzyl ester

To a solution of 3.1 g of 4-(benzyloxycarbonyl-methyl-amino)-butyric acid in 80 mL DCM were added 6.5 mL of DIPEA, 1.8 g of HOBt, 2.6 g of EDC and 154 mg of DMAP. After stirring for 10 min, 2.1 g of 3,6-dimethoxy-benzene-1,2-diamine, dissolved in 20 mL DCM, were added and the mixture was stirred at rt overnight. The reaction was quenched with sat. aq. NaHCO₃, the phases were separated and the organic phase was washed with brine, dried over MgSO₄ and concentrated in vacuo to yield the crude title compound as black oil.

LC-MS: t_{R}=0.88 min; [M+H]⁺: 402.06.

### BB.3 [3-(4,7-Dimethoxy-1H-benzoimidazol-2-yl)-propyl]-methyl-carbamic acid benzyl ester

To a mixture of the above crude [3-(2-amino-3,6-dimethoxy-phenylcarbamoyl)-propyl]-methyl-carbamic acid benzyl ester in 16 mL toluene were added 4 mL of DMF and 1.9 g of TsOH and the reaction was heated to 150° C. for 2 h in the microwave. Sat. aq. NaHCO₃ was added and the phases were separated. The organic phase was washed with brine, dried over MgSO₄, concentrated in vacuo, filtered over a short pad of silica gel with EtOAc and concentrated again. Purification by CC with EtOAc yielded 2.7 g of 3-(4,7-dimethoxy-1H-benzoimidazol-2-yl)-propyl]-methyl-carbamic acid benzyl ester as brown resin.

LC-MS: t_{R}=0.85 min; [M+H]⁺: 384.62.

BB.4 [3-(4,7-Dimethoxy-1H-benzoimidazol-2-yl)-propyl]-methyl-amine

A solution of 2.6 g of 3-(4,7-dimethoxy-1H-benzoimidazol-2-yl)-propyl]-methyl-carbamic acid benzyl ester in 60 mL EtOH was evacuated 3 times with N₂ before 260 mg of 10 wt % Pd/C were added. The reaction mixture was then stirred under a H₂ atmosphere (balloon) for 5 h at rt. Filtration over a pad of celite and washing with EtOH yielded after concentration in vacuo 1.7 g of 3-(4,7-dimethoxy-1H-benzoimidazol-2-yl)-propyl]-methyl-amine as brown foam.

LC-MS: t_{R}=0.57 min; [M+H]⁺: 250.13.

### PREPARATION OF EXAMPLES

### Example 1

### rac-(1R*,2R*,4R*)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol

### 1.1 (Procedure P1.1): rac-(1R*,2R*,4R*)-(2-Hydroxy-5-phenyl-bicyclo[2.2.2]oct-5-en-2-yl)-acetic acid

To a solution of 4.0 g of rac-(1R*,2R*,4R*)-(2-hydroxy-5-phenyl-bicyclo[2.2.2]oct-5-en-2-yl)-acetic acid tert.-butyl ester in 25 mL EtOH were added 2.1 g of LiOH.H₂O, 8 mL H₂O and 22 mL MeOH. The reaction mixture was stirred at rt for 3 d and then concentrated. The residue was partitioned between water and Et₂O. The aq. layer was separated and acidified with 1N HCl resulting in the formation of a white solid. The solid was filtrated, washed with 5 mL dil. HCl and dried in vacuo to obtain 3.2 g of rac-(1R*,2R*,4R*)-(2-hydroxy-5-phenyl-bicyclo[2.2.2]oct-5-en-2-yl)-acetic acid as white solid.

LC-MS: t_{R}=0.86 min; [M−H₂O+H]⁺: 241.28.

### 1.2 (Procedure P1.2): rac-(1R*,2R*,4R*)-N-[3-(4,7-Dimethoxy-1H-benzoimidazol-2-yl)-propyl]-2-(2-hydroxy-5-phenyl-bicyclo[2.2.2]oct-5-en-2-yl)-N-methyl-acetamide

To a solution of 280 mg of rac-(1R*,2R*,4R*)-(2-hydroxy-5-phenyl-bicyclo[2.2.2]oct-5-en-2-yl-acetic acid in 7 mL THF were added 0.58 mL of DIPEA, 175 mg of HOBt and 250 mg of EDC at rt. After stirring for 10 min, 270 mg of 3-(4,7-dimethoxy-1H-benzoimidazol-2-yl)-propyl]-methyl-amine were added and the reaction mixture was stirred at rt overnight. The reaction mixture was quenched with sat. aq. NaHCO₃, the phases were separated and the organic phase was washed with water and brine, dried over MgSO₄ and concentrated in vacuo. Purification by CC using EtOAc-MeOH (5:1 to 2:1) yielded 475 mg of rac-(1R*,2R*,4R*)-N-[3-(4,7-dimethoxy-1H-benzoimidazol-2-yl)-propyl]-2-(2-hydroxy-5-phenyl-bicyclo[2.2.2]oct-5-en-2-yl)-N-methyl-acetamide as white foam.

LC-MS: t_{R}=0.91 min; [M+H]⁺: 490.06.

### 1.3 (Procedure P1.3): rac-(1R*,2R*,4R*)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol

To a solution of 310 mg of rac-(1R*,2R*,4R*)-N-[3-(4,7-dimethoxy-1H-benzoimidazol-2-yl)-propyl]-2-(2-hydroxy-5-phenyl-bicyclo[2.2.2]oct-5-en-2-yl)-N-methyl-acetamide in 8 mL toluene were added dropwise 0.77 mL of a Red-Al solution (65% in toluene) at 0° C. After stirring for 10 min at 0° C., the cooling bath was removed and stirring was continued for 3 h at rt. The reaction mixture was then carefully poured onto a mixture of 1 M NaOH/ice and stirred for 10 min. The aq. phase was extracted with toluene, the combined organic phases were washed with brine, dried over MgSO₄ and concentrated in vacuo. Purification by CC using EtOAc-MeOH (2:1) yielded 230 mg of rac-(1R*,2R*,4R*)-2-(2-{[3-(4,7-dimethoxy-1H-benzoimidazol-2-yl)-propyl]-methyl-amino}-ethyl)-5-phenyl-bicyclo[2.2.2]oct-5-en-2-ol as white foam.

LC-MS: t_{R}=0.79 min; [M+H]⁺: 476.13.

### Example 1A

### rac-Isobutyric acid (1R*,2R*,4R*)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester

### 1A.1 (Procedure P1.4): rac-Isobutyric acid (1R*,2R*,4R*)-2-(2-{[3-(4,7-dimethoxy-1H-benzoimidazol-2-0)-propyl]-methyl-amino}-ethyl)-5-phenyl-bicyclo[2.2.2]oct-5-en-2-yl ester

To a solution of 199 mg of rac-(1R*,2R*,4R*)-2-(2-{[3-(4,7-dimethoxy-1H-benzoimidazol-2-yl)-propyl]-methyl-amino}-ethyl)-5-phenyl-bicyclo[2.2.2]oct-5-en-2-ol in 4 mL DCM were added 0.2 mL of NEt₃ and 0.1 mL of isobutyrylchloride at 0° C. The reaction mixture was stirred overnight allowing the temperature to reach slowly rt. The reaction was quenched with sat. aq. NaHCO₃, the phases were separated and the water phase was reextracted with DCM. The combined organic phases were washed with brine, dried over MgSO₄ and concentrated in vacuo. The residue was redissolved in 3 mL EtOAc, silica gel and 1.5 mL MeOH were added and the mixture was stirred vigorously for 7 d. The mixture was filtered, thoroughly washed with EtOAc-MeOH (2:1) and evaporated. Purification by CC using EtOAc-MeOH (5:1 to 3:1+0.1% NEt3) yielded 186 mg of rac-isobutyric acid (1R*,2R*,4R*)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester as beige foam.

LC-MS: t_{R}=0.90 min; [M+H]⁺: 546.23.

### 1A.2 (Procedure P1.5): rac-Isobutyric acid (1R*,2R*,4R*)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester dihydrochloride

The above product may be transformed into the corresponding dihydrochloride salt using the following procedure.

To a solution of 186 mg of rac-isobutyric acid (1R*,2R*,4R*)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester in 2 mL EtOAc were added 0.3 mL of 3M HCl in EtOAc at 0° C. The reaction mixture was evaporated to dryness without heating to give 199 mg of rac-isobutyric acid (1R*,2R*,4R*)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester as dihydrochloride.

### Example 2

### (1R,2R,4R)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol or (1S,2S,4S)-2-(2-{[3-(4,7-dimethoxy-1H-benzoimidazol-2-yl)-propyl]-methyl-amino}-ethyl)-5-phenyl-bicyclo[2.2.2]oct-5-en-2-ol

### 2.1: (1R,2R,4R)-(2-Hydroxy-5-phenyl-bicyclo[2.2.2]oct-5-en-2-0)-acetic acid or (1S,2S,4S)-(2-hydroxy-5-phenyl-bicyclo[2.2.2]oct-5-en-2-0)-acetic acid

Prepared according to procedure P1.1 in Example 1 using enantiomer B of rac-(1R*,2R*,4R*)-(2-hydroxy-5-phenyl-bicyclo[2.2.2]oct-5-en-2-yl)-acetic acid tert.-butyl ester (see K1A.6).

LC-MS: t_{R}=0.91 min; [M−H₂O+H]⁺: 241.10.

### 2.2: (1R,2R,4R)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol or (1S,2S,4S)-2-(2-{[3-(4,7-dimethoxy-1H-benzoimidazol-2-yl)-propyl]-methyl-amino}-ethyl)-5-phenyl-bicyclo[2.2.2]oct-5-en-2-ol

Prepared according to procedures P1.2 to P1.3 in Example 1 using the above (2-hydroxy-5-phenyl-bicyclo[2.2.2]oct-5-en-2-yl)-acetic acid.

LC-MS: t_{R}=0.78 min; [M+H]⁺: 476.09.

### Example 2A

### Isobutyric acid (1R,2R,4R)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester or isobutyric acid (1S,2S,4S)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester

Prepared according to procedure P1.4 in Example 1A using the above 2-(2-{[3-(4,7-dimethoxy-1H-benzoimidazol-2-yl)-propyl]-methyl-amino}-ethyl)-5-phenyl-bicyclo[2.2.2]oct-5-en-2-ol (compound of example 2).

LC-MS: t_{R}=0.89 min; [M+H]⁺: 546.19.

### Example 3

### (1R,2R,4R)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol or (1S,2S,4S)-2-(2-{[3-(4,7-dimethoxy-1H-benzoimidazol-2-yl)-propyl]-methyl-amino}-ethyl)-5-phenyl-bicyclo[2.2.2]oct-5-en-2-ol

### 3.1: (1R,2R,4R)-(2-Hydroxy-5-phenyl-bicyclo[2.2.2]oct-5-en-2-0)-acetic acid or (1S,2S,4S)-(2-hydroxy-5-phenyl-bicyclo[2.2.2]oct-5-en-2-0)-acetic acid

Prepared according to procedure P1.1 in Example 1 using enantiomer A of rac-(1R*,2R*,4R*)-(2-hydroxy-5-phenyl-bicyclo[2.2.2]oct-5-en-2-yl)-acetic acid tert.-butyl ester (see K1A.6).

LC-MS: t_{R}=0.91 min; [M−H₂O+H]⁺: 241.16.

### 3.2: (1R,2R,4R)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol or (1S,2S,4S)-2-(2-{[3-(4,7-dimethoxy-1H-benzoimidazol-2-yl)-propyl]-methyl-amino}-ethyl)-5-phenyl-bicyclo[2.2.2]oct-5-en-2-ol

Prepared according to procedures P1.2 to P1.3 in Example 1 using the above (2-hydroxy-5-phenyl-bicyclo[2.2.2]oct-5-en-2-yl)-acetic acid.

LC-MS: t_{R}=0.79 min; [M+H]⁺: 476.09.

### Example 3A

### Isobutyric acid (1R,2R,4R)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester or isobutyric acid (1S,2S,4S)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester

Prepared according to procedure P1.4 in Example 1A using the above 2-(2-{[3-(4,7-dimethoxy-1H-benzoimidazol-2-yl)-propyl]-methyl-amino}-ethyl)-5-phenyl-bicyclo[2.2.2]oct-5-en-2-ol (compound of example 3).

LC-MS: t_{R}=0.89 min; [M+H]⁺: 546.11.

### Example 4

### rac-(1R*,5R*,6R*)-6-(2-{[3-(4,7-Dimethoxy-1H-benzoimidazol-2-yl)-propyl]-methyl-amino}-ethyl)-8-phenyl-bicyclo[3.2.2]non-8-en-6-ol

### 4.1: rac-(1R*,5R*,6R*)-(6-Hydroxy-8-phenyl-bicyclo[3.2.2]non-8-en-6-yl)-acetic acid

Prepared according to procedure P1.1 in Example 1 using rac-(1R*,5R*,6R*)-6-hydroxy-8-phenyl-bicyclo[3.2.2]non-8-en-6-yl)-acetic acid tert.-butyl ester (see K2A.3).

LC-MS: t_{R}=0.96 min; [M+Na+H]⁺: 296.10.

### 4.2: rac-(1R*,5R*,6R*)-6-(2-{[3-(4,7-Dimethoxy-1H-benzoimidazol-2-yl)-propyl]-methyl-amino}-ethyl)-8-phenyl-bicyclo[3.2.2]non-8-en-6-ol

Prepared according to procedures P1.2 to P1.3 in Example 1 using rac-(1R*,5R*,6R*)-(6-hydroxy-8-phenyl-bicyclo[3.2.2]non-8-en-6-yl)-acetic acid.

LC-MS: t_{R}=0.80 min; [M+H]⁺: 490.06.

### Example 4A

### rac-Isobutyric acid (1R*,5R*,6R*)-6-(2-{[3-(4,7-dimethoxy-1H-benzoimidazol-2-yl)-propyl]-methyl-amino}-ethyl)-8-phenyl-bicyclo[3.2.2]non-8-en-6-yl ester

Prepared according to procedure P1.4 in Example 1A using rac-(1R*,5R*,6R*)-6-(2-{[3-(4,7-dimethoxy-1H-benzoimidazol-2-yl)-propyl]-methyl-amino}-ethyl)-8-phenyl-bicyclo[3.2.2]non-8-en-6-ol.

LC-MS: t_{R}=0.91 min; [M+H]⁺: 560.05.

Biological tests

### In Vitro Assay L Channel

The L channel antagonistic activity (IC₅₀ values) of the compounds of formula (I) is determined in accordance with the following experimental method.

Human embryonic kidney (HEK293) cells expressing the human Caᵥ1.2 channel in addition to the auxiliary subunits β-2a and α2δ-1, are grown in culture medium (DMEM containing 10% heat-inactivated fetal calf serum (FCS), 100 U/ml penicillin, 100 μg/ml streptomycin, 100 μg/ml G418, 40 μg/ml zeocin and 100 μg/ml hygromycin). The cells are seeded at 20,000 cells/well into 384-well black clear bottom sterile plates (poly-L-lysine-coated, Becton Dickinson). The seeded plates are incubated overnight at 37° C. in 5% CO₂. The KCl solution is prepared as 80 mM stock solution in assay buffer (HBSS containing 0.1% BSA, 20 mM HEPES, 0.375 g/l NaHCO₃, adjusted to pH 7.4 with NaOH) for use in the assay at a final concentration of 20 mM. Antagonists are prepared as 10 mM stock solutions in DMSO, then diluted in 384w plates first in DMSO, then in assay buffer to obtain 3× stocks. On the day of the assay, 25 μl of staining buffer (HBSS containing 20 mM HEPES, 0.375 g/l NaHCO₃, and 30 μM of the fluorescent calcium indicator fluo-4 AM (1 mM stock solution in DMSO, containing 10% pluronic) is added to each well of the seeded plate. The 384-well cell-plates are incubated for 60 min at 37° C. in 5% CO₂ followed by washing with 2×50 ml per well using assay buffer leaving 50 ml/well of this buffer for equilibration at room temperature (30-60 min). Within the Fluorescent Imaging Plate Reader (FLIPR, Molecular Devices), antagonists are added to the plate in a volume of 25 μl/well, incubated for 3 min and finally 25 μl/well of KCl solution is added for cellular depolarization. Fluorescence is measured for each well at 2 second intervals for 8 minutes, and the area under the curve of each fluorescence peak is compared to the area of the fluorescence peak induced by 20 mM KCl with vehicle in place of antagonist. For each antagonist, the IC₅₀ value (the concentration (in nM) of compound needed to inhibit 50% of the KCl-induced fluorescence response) up to 10 mM is determined.

Compounds of examples 1, 2, 3, 4 have not been tested in this assay. IC₅₀ values of example compounds IA, 2A, 3A and 4A are in the range of 156 to 439 nM with an average of 305 nM.

### In Vitro Assay T Channel:

The T channel antagonistic activity (IC₅₀ values) of the compounds of formula (I) is determined in accordance with the following experimental method and data are shown in Table 1.

Human embryonic kidney (HEK293) cells expressing the human Caᵥ3.1 Caᵥ3.2 or Caᵥ3.3 channel, respectively, are grown in culture medium (DMEM containing 10% heat-inactivated fetal calf serum (FCS), 100 U/ml penicillin, 100 μg/ml streptomycin and 1 mg/ml G418). The cells are seeded at 20,000 cells/well into 384-well black clear bottom sterile plates (poly-L-lysine-coated, Becton Dickinson). The seeded plates are incubated overnight at 37° C. in 5% CO₂. The Ca²⁺solution is prepared as 100 mM stock solution in 100 mM tetraethylammoniumchloride (TEA-chloride), 50 mM HEPES, 2.5 mM CaCl₂, 5 mM KCl, 1 mM MgCl₂, adjusted to pH 7.2 with TEA-hydroxide, for use in the assay at a final concentration of 10 mM. Antagonists are prepared as 10 mM stock solutions in DMSO, then diluted in 384w plates first in DMSO, then in 100 mM TEA-chloride, 50 mM HEPES, 2.5 mM CaCl₂, 5 mM KCl, 1 mM MgCl₂, adjusted to pH 7.2 with TEA-hydroxide, to obtain 9× stocks. On the day of the assay, 25 μl of staining buffer (HBSS containing 20 mM HEPES, 0.375 g/l NaHCO₃ and 30 μM of the fluorescent calcium indicator fluo-4 AM (1 mM stock solution in DMSO, containing 10% pluronic) is added to each well of the seeded plate. The 384-well cell-plates are incubated for 60 min at 37° C. in 5% CO₂ followed by washing with 2×50 ml per well using HBSS containing 0.1% BSA, 20 mM HEPES, 0.375 g/l NaHCO₃, leaving 50 ml/well of this buffer for equilibration at room temperature (30-60 min). Within the Fluorescent Imaging Plate Reader (FLIPR, Molecular Devices), antagonists are added to the plate in a volume of 6.25 ml/well, incubated for 3 min, and finally 6.25 ml/well of Ca²⁺solution is added. Fluorescence is measured for each well at 2 second intervals for 8 minutes, and the area under the curve of each fluorescence peak is compared to the area of the fluorescence peak induced by 10 mM Ca²⁺with vehicle in place of antagonist. For each antagonist, the IC₅₀ value (the concentration (in nM) of compound needed to inhibit 50% of the Ca²⁺-induced fluorescence response) up to 10 mM is determined.

TABLE 1

| Compound | IC₅₀ |
|---|---|
| 1 | NA |
| 1A | 571 |
| 2 | NA |
| 2A | 778 |
| 3 | NA |
| 3A | 793 |
| 4 | NA |
| 4A | 727 |

NA = not available/not tested

### Effect on Isolated Hearts According to the Langendorff Method (Lgdff)

The compounds were tested for their potential to reduce blood pressure and their effect on the contractility of the heart muscle. EC₅₀ values on isolated mouse hearts were determined according to Literature (Doring H J., The isolated perfused heart according to Langendorff technique—function—application, Physiol. Bohemoslov. 1990, 39(6), 481-504; Kligfield P, Horner H, Brachfeld N., A model of graded ischemia in the isolated perfused rat heart, J. Appl. Physiol. 1976 June, 40(6), 1004-8).

The compound of example 1A has been measured using the procedure described above for the Langendorff experiment with an EC₅₀ of 5 nM.

1. A compound of the formula (I) 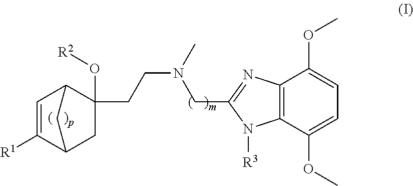
wherein
R¹ represents aryl, which is unsubstituted, or mono-, di-, or tri-substituted wherein the substituents are independently selected from the group consisting of (C₁₋₄)alkyl, (C₁₋₄)alkoxy, halogen, and trifluoromethyl;
R² represents hydrogen, or —CO—R²¹;
R²¹ represents (C₁₋₅)alkyl, (C₁₋₃)fluoroalkyl, or (C₃₋₆)cycloalkyl;
m represents the integer 2, or 3;
p represents the integer 2 or 3; and
R³ represents hydrogen, or (C₁₋₆)alkyl;
or a pharmaceutically acceptable salt thereof.

2. A compound according to claim 1, wherein the configuration of the bridged cyclohexene moiety is such that the R²—O— substituent and the bridge —(CH₂)ₚ— of the cyclohexene moiety are in cis relation;
or a pharmaceutically acceptable salt thereof.

3. A compound according to claim 1, wherein
R¹ represents unsubstituted phenyl;
or a pharmaceutically acceptable salt thereof.

4. A compound according to claim 1, wherein
R² represents —CO—R²¹;
or a pharmaceutically acceptable salt thereof.

5. A compound according to claim 1, wherein
R²¹ represents (C₁₋₅)alkyl;
or a pharmaceutically acceptable salt thereof.

6. A compounds of according to claim 1, wherein
m represents the integer 3;
or a pharmaceutically acceptable salt thereof.

7. A compounds of according to claim 1, wherein
R³ represents hydrogen;
or a pharmaceutically acceptable salt thereof.

8. A compound according to claim 1, selected from the following compounds:
Isobutyric acid (1R,2R,4R)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester;
Isobutyric acid (1S,2S,4S)-2-(2-{[3-(4,7-dimethoxy-1H-benzoimidazol-2-yl)-propyl]-methyl-amino}-ethyl)-5-phenyl-bicyclo[2.2.2]oct-5-en-2-yl ester;
Isobutyric acid (1R,5R,6R)-6-(2-{[3-(4,7-dimethoxy-1H-benzoimidazol-2-yl)-propyl]-methyl-amino}-ethyl)-8-phenyl-bicyclo[3.2.2]non-8-en-6-yl ester; and
Isobutyric acid (1S,5S,6S)-6-(2-{[3-(4,7-dimethoxy-1H-benzoimidazol-2-yl)-propyl]-methyl-amino}-ethyl)-8-phenyl-bicyclo[3.2.2]non-8-en-6-yl ester;
or a pharmaceutically acceptable salt thereof.

9. A compound according to claim 1, selected from the following compounds:
(1R,2R,4R)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol;
(1S,2S,4S)-2-(2-{[3-(4,7-Dimethoxy-1H-benzoimidazol-2-yl)-propyl]-methyl-amino}-ethyl)-5-phenyl-bicyclo[2.2.2]oct-5-en-2-ol;
(1R,5R,6R)-6-(2-{[3-(4,7-Dimethoxy-1H-benzoimidazol-2-yl)-propyl]-methyl-amino}-ethyl)-8-phenyl-bicyclo[3.2.2]non-8-en-6-ol; and
(1S,5S,6S)-6-(2-{[3-(4,7-Dimethoxy-1H-benzoimidazol-2-yl)-propyl]-methyl-amino}-ethyl)-8-phenyl-bicyclo[3.2.2]non-8-en-6-ol;
or a pharmaceutically acceptable salt thereof.

10. A pharmaceutical composition comprising a compound according to claim 1, or a pharmaceutically acceptable salt thereof, and a pharmaceutically acceptable carrier.

11. A pharmaceutical composition according to claim 10, further comprising at least one therapeutically inert excipient.

12. A method for the treatment or prophylaxis of a disease selected from chronic stable angina, hypertension, ischemia (renal and cardiac), cardiac arrhythmias including atrial fibrillation, cardiac hypertrophy, or congestive heart failure comprising administering to a patient in need thereof the composition of claim 1.

13. (canceled)
